SEQUENCE LISTING (5 sequences)

SEQ ID NO: 1 - DNA, 20 bp, synthetic construct
  source 1..20: mol_type = other DNA
ttacatggaaagctcatctt

SEQ ID NO: 2 - DNA, 20 bp, synthetic construct
  source 1..20: mol_type = other DNA
ttacgaatgccttggaaacc

SEQ ID NO: 3 - DNA, 20 bp, synthetic construct
  source 1..20: mol_type = other DNA
gcatctgaagaccgatgatc

SEQ ID NO: 4 - DNA, 20 bp, synthetic construct
  source 1..20: mol_type = other DNA
cttgatacaggtttaattcg

SEQ ID NO: 5 - DNA, 20 bp, synthetic construct
  source 1..20: mol_type = other DNA
cacgcacctggacagctgac

(19) United States
(12) Patent Application Publication
(10) Pub. No.: US 2024/0408201 A1
(43) Pub. Date: Dec. 12, 2024

(54) CONVERSION-RESISTANT / CONDITION-RESISTANT TREGS AND CAR TREGS, METHODS OF MAKING AND METHODS OF USING

(71) Applicant: The Trustees of Columbia University in the City of New York, New York, NY (US)

(72) Inventors: Mohsen Khosravi MAHARLOOEI, Fort Lee, NJ (US); Remi CREUSOT, New York, NY (US); Megan SYKES, New York, NY (US)

(21) Appl. No.: 18/814,036

(22) Filed: Aug. 23, 2024

Related U.S. Application Data
(63) Continuation of application No. PCT/US2023/064413, filed on Mar. 15, 2023.
(60) Provisional application No. 63/320,008, filed on Mar. 15, 2022.

(51) Int. Cl. A61K 39/00; A61P 37/06; C07K 16/28; C12N 5/0783; C12N 9/22; C12N 15/11; C12N 15/90

(52) U.S. Cl. CPC A61K 39/4611; A61K 39/4631; A61K 39/4643; A61P 37/06; C07K 16/2833; C12N 5/0637; C12N 9/22; C12N 15/11; C12N 15/907; C07K 2317/73; C12N 2310/20; C12N 2510/00; C12N 2800/80

(57) ABSTRACT

CAR Tregs and Tregs are provided which are both conversion-resistant and condition-resistant. The Treg cells are engineered such that they are deficient in or substantially devoid of a cell-surface marker or antigen.

### CROSS-REFERENCE TO RELATED APPLICATIONS

This application is a continuation of PCT International Application No. PCT/US2023/064413, filed Mar. 15, 2023, which claims priority to U.S. Provisional Patent Application No. 63/320,008, filed Mar. 15, 2022, each of which is hereby incorporated by reference in its entirety.

### STATEMENT OF GOVERNMENT SUPPORT

This invention was made with government support under grant numbers DK123559 and AI045897 awarded by the National Institutes of Health. The government has certain rights in this invention.

### REFERENCE TO SEQUENCE LISTING

This application incorporates-by-reference nucleotide sequences which are present in the file named “2024-08-23_93597-7196_92231-A-PCT-A_Sequence_Listing_AWG.xml”, which is 5,547 bytes in size, and which was created on Aug. 23, 2024 in the IBM-PC machine format, having an operating system compatibility with MS-Windows, which is contained in the xml file filed Aug. 23, 2024 as part of this application.

### BACKGROUND

There is a need for a safer and more effective immunosuppressive therapy for transplantation and autoimmunity. Improved and targeted immunosuppressive therapy could reduce the risk of organ rejection and side effects from general immunosuppression. It could also open the door for an alternative source of organs to address the organ shortage.

Regulatory T (Treg) cells are immunosuppressive and generally suppress or downregulate induction and proliferation of effector T (Teff) cells. Tregs prevent immune responses to non-pathogenic antigens and are the primary modulators of peripheral tolerance. Treg cells can be tuned to tolerate select antigens through exposure to these stimuli in vivo or ex vivo.

Chimeric antigen receptor or CAR Treg cell therapy is promising for preventing and treating autoimmune diseases and promoting immunologic tolerance in transplantation by recognizing non-threatening antigens. However, clinical implementation of Treg cell therapy is hindered by multiple factors, including CAR Treg plasticity and inefficient trafficking to target organs and their draining lymph nodes. As CAR Tregs are specific for self-antigens, they could have detrimental effects if they lose their regulatory phenotype and convert to effector CAR T cells, i.e., conversion. Thus, despite promising results in using CAR Tregs for tolerance induction, there are still major considerations regarding the plasticity of CAR Tregs (Zhou et al. 2009; Koenen et al. 2008) and the effect of T cell-depleting conditioning regimens (Neclapu et al. 2019) on these cells that need to be addressed before they are used clinically. See also Raffin et al. 2020. Inflammation is also shown to drive Treg conversion (Hua et al. 2018). CAR Tregs can quickly reject the target organs if they lose their regulatory phenotype and convert to Teff.

Thus, there is a need for CAR Tregs that are both condition-resistant and conversion-resistant as well as a simple method to obtain these cells.

### SUMMARY

Disclosed herein are CAR Tregs which are both conversion-resistant, i.e., resistant conversion to effector T cells, and condition-resistant, i.e., resistant to a T cell-depleting conditioning regimen involving an antibody, in some instances an anti-CD2 antibody. The disclosed CAR Tregs can comprise a first nucleic acid construct encoding a chimeric antigen receptor (CAR) and a Treg cell that is engineered such that it is deficient in or substantially devoid of a cell-surface marker or antigen, i.e., the entire or portion of the gene encoding the cell-surface marker or antigen is deleted from the Treg cell.

Also disclosed herein are regulatory T (Treg) cells that are engineered such that it is deficient in or substantially devoid of a cell-surface marker or antigen, e.g., CD2, i.e., the entire or portion of the gene encoding the cell-surface marker or antigen, e.g., CD2, is deleted from the Treg cells. These CD2− cells have increased functionality as compared CD2+ Treg cells and are both conversion-resistant and condition-resistant.

Also disclosed herein is a one-step method for obtaining such conversion-resistant/condition-resistant CAR Tregs and Tregs, wherein the only manipulation of the cell is removing or inhibiting a cell-surface molecule or antigen, in some instances CD2, using one type of engineering. Such a method has great advantage over other multiple step methods, and methods which alter a cell in more than one fashion as it is always beneficial to manipulate and/or modify cells as little as possible.

A method utilizing CRISPR technology to remove a cell-surface marker, e.g., CD2 molecule, from CAR Tregs genome can be used to make CAR Tregs resistant to a T cell-depleting conditioning regimen involving an antibody, e.g., an anti-CD2 antibody. This removal will ensure that the Tregs are not affected by an antibody being used to deplete recipient T cells in order to open space for the adoptively-transferred CAR Tregs. However, as disclosed herein, the removal of the CD2 molecule unexpectedly also made the cells conversion-resistant, as well as more potent in suppressing immune responses than CAR Treg cells which were CD2+.

Thus, the present disclosure provides methods of producing the conversion-resistant/condition-resistant CAR Tregs or Tregs. In some embodiments, the method utilizes recombinant or genetic engineering techniques. In some embodiments, the method utilizes CRISPR technology. In some embodiments, the method of producing the conversion-resistant/condition-resistant CAR Tregs is one step using one type of engineering. In some embodiments, the one step involves the removal or inhibition of a cell-surface marker or antigen from the cells. In some embodiments, the entire or a portion of the gene encoding the cell-surface marker or antigen is deleted from the Treg cell. In some embodiments, the cell-surface antigen is CD2. In some embodiments, the removal or inhibition is achieved utilizing CRISPR technology.

In some embodiments, the disclosure provides for methods for removing or inhibiting a cell-surface marker or antigen in a Treg cell comprising introducing into the cell: (i) at least one guide RNA (gRNA) or DNA encoding at least one guide RNA (gRNA); and (ii) at least one RNA-guided endonuclease or nucleic acid encoding an RNA-guided endonuclease. In some embodiment, the RNA-guided endonuclease is a Cas nuclease. In some embodiments, the Cas nuclease is Cas9. In some embodiments, the RNA-guided endonuclease and gRNA are introduced into the cell in the form of a ribonucleoprotein complex comprising the endonuclease complexed to least one gRNA. Preparation of such RNP complexes is known in the art or can be obtained commercially. In some embodiments, the gRNA has a nucleotide sequence selected from the group consisting of SEQ ID NOs: 1-5.

In some embodiments, the present disclosure provides for a Treg cell comprising a first nucleic acid construct encoding a CAR. The CAR comprises an antigen-binding region.

In some embodiments, the CAR binds to human leukocyte antigen A2 (HLA-A2).

In some embodiments, the CAR may be operably linked to a Treg-specific promoter including but not limited to the forkhead box P3 (Foxp3) promoter. Other Treg-specific genes include but are not limited to peptidase inhibitor 16 (Sadlon et al. 2010).

The antigen-binding region of the CAR may be a single-chain variable fragment (scFv) comprising a light chain variable region (VL) and a heavy chain variable region (VH).

In some embodiments, the CAR comprises a cytoplasmic signaling domain of CD3ζ.

The present disclosure provides for conversion-resistant/conditioning-resistant CAR Tregs or Tregs for induction of immune tolerance in transplantation and for treating and/or preventing autoimmune diseases and disorders.

The present disclosure also provides for compositions, including pharmaceutical composition, comprising the cells disclosed herein.

The present disclosure also provides for methods of using the disclosed cells and compositions.

The present cells and compositions may be used in various therapeutic, prophylactic, diagnostic and other methods. The present cells and compositions and methods may be used in any situation in which immunosuppression is desired, e.g., transplant rejection or autoimmune diseases and disorders. The present cells and compositions and methods may be used to reduce complications associated with organ or tissue transplantation, reduce the likelihood of transplant rejection, prevent transplant rejection, treat transplant rejection, and induce immune tolerance. The present cells and composition and methods may be used to treat and/or prevent an autoimmune disease and disorder. The present cells and composition and methods may be used to treat and/or prevent graft-versus-host disease. The compositions contain the disclosed cells.

The present Treg cells may be used to suppress rejection responses to donor organs where the specific antigens are unknown.

The present disclosure also provides for kits comprising cells, compositions and pharmaceutical compositions disclosed herein, as well as kits for producing the disclosed cells.

### BRIEF DESCRIPTION OF THE FIGURES

For the purpose of illustrating the invention, there are depicted in drawings certain embodiments of the invention. However, the invention is not limited to the precise arrangements and instrumentalities of the embodiments depicted in the drawings.

FIG. 1 is a schematic of the general method to remove CD2.

FIGS. 2A-2B show the removal of CD2 using CRISPR and five different gRNAs. FIG. 2A are representative flow cytometry images of the CD4 T cells with CD2 removed using guide RNAs FIG. 2A-1: RNAs #1-#2 and FIG. 2A-2: RNAs #3-#5. FIG. 2B is a graph of cell counts of the T cells after CRISPR treatment with each guide RNA. The removal of CD2 slowed down T cell proliferation but the cells with CD2 removed using any of the guide RNAs were still capable of expanding to high numbers.

FIGS. 3A-3B show representative flow cytometry images of Treg cells, both 3A) untreated (no removal of CD2, designated “CD+”) and 3B) treated (removal of CD2, designated “CD-KO”) and both CAR+ and CAR−.

FIGS. 4A-4D are graphs quantifying the flow cytometry of FIG. 3. FIG. 4A shows Tregs stimulated with DC #1. FIG. 4B shows Tregs stimulated with DC #2. FIG. 4C shows CAR Tregs stimulated with DC #1. FIG. 4D shows CAR Tregs stimulated with DC #2. The darker lines in FIGS. 4A-4D represent CD2+ cells. The lighter lines in FIGS. 4A-4D represent CD-KO cells. The in vitro results in FIGS. 3 and 4 show that the removal CD2 from the Tregs increased both stability and functionality of the Tregs in suppressing T cell responses.

FIG. 5 is a schematic of the in vivo model for testing the CD2− CAR Tregs and CD2− Tregs.

FIGS. 6A-6C show the results of the in vivo testing on mice injected with four different Tregs and a control group. FIG. 6A is a survival curve of all groups. FIG. 6B shows the GVHD scores in each group. FIG. 6C shows the percent of weight change in each group. Legend: No Tregs-line with flattened circles; No CAR/CD2+ Tregs—dark line with circles; No CAR/CD2-KO Tregs-light line with squares; HLA-A2 CAR/CD2+ Tregs—light line with triangles; HLA-A2 CAR/CD2-KO Tregs-light line with diamonds.

FIGS. 7A-7D show the results of the analysis of immune cells in peripheral blood using flow cytometry. FIG. 7A shows the percentage of CD3 cells among hCD45 cells at weeks 1, 2, and 4. FIG. 7B shows the percentage of CD8 cells among CD3 cells at weeks 1, 2, and 4. FIG. 7C shows the hCD3 cell count per 50 μl of blood at weeks 1, 2, and 4. FIG. 7D shows the CD8 cell count per 50 μl of blood at weeks 1, 2, and 4. Legend: No Tregs-line with flattened circles; No CAR/CD2+ Tregs—dark line with circles; No CAR/CD2-KO Tregs-light line with squares; HLA-A2 CAR/CD2+ Tregs—light line with triangles; HLA-A2 CAR/CD2-KO Tregs-light line with diamonds. The in vivo results in FIGS. 6 and 7 show that the CD2− Tregs and the CD2− CAR Tregs had higher functionality in preventing the expansion of T cells and delaying the development of GVHD than CD2+ Tregs and CD2+ CAR Tregs.

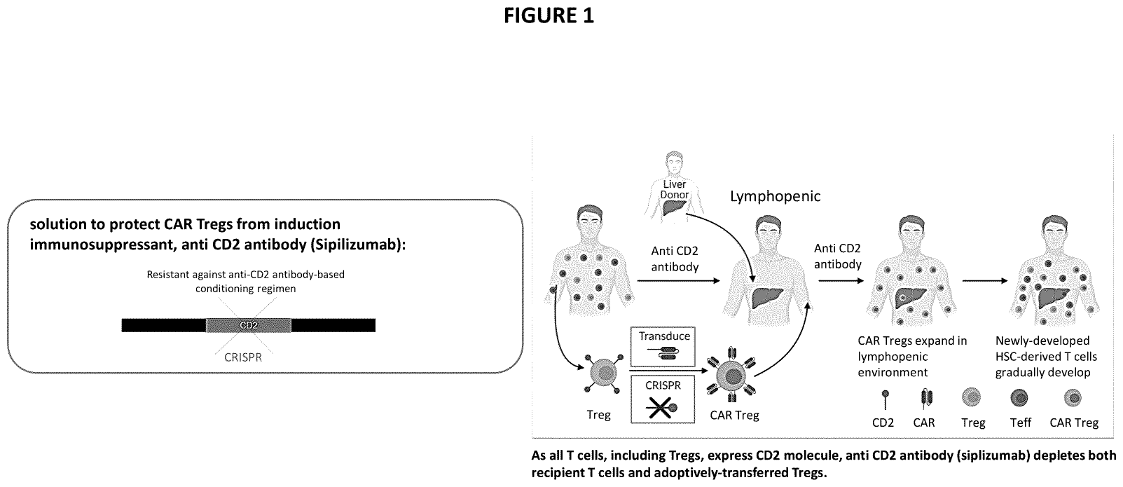

### DETAILED DESCRIPTION

The present disclosure provides for chimeric antigen receptor or CAR Treg cells as well as Treg cells, which are both conversion-resistant and condition-resistant, to induce immune tolerance in transplantation and for treating and/or preventing autoimmune diseases. The present cells, compositions and methods prevent transformation of CAR Tregs and Tregs to effector T (Teff) cells as well as making them condition-resistant to an antibody conditioning regimen, such as an anti-CD2 antibody regimen. The present disclosure also provides for a method to obtain the cells which is one step involving the removal or inhibition of a cell-surface marker, e.g., CD2, from the Treg, using CRISPR.

This is a much simpler method, involving only one type of engineering, to obtain conversion-resistant/condition resistant CAR Tregs and Tregs cells with increased functionality. The less manipulation of the cells is beneficial. In general, due to the off-target effects of any engineering procedure, it is preferred to limit the number of genetic engineering targets for clinical applications. Thus, if removing one molecule, e.g., CD2, can provide two benefits at the same time, it would be preferred to be used in clinical settings. Additionally, one step is easier and less likely to be subject to user error when performed by laboratory or clinical personnel.

The present disclosure also provides for a Treg cell which is deficient in or substantially devoid of a cell-surface marker, including but not limited to CD2.

In some embodiments, the Treg cell comprises a first nucleic acid construct encoding a CAR. The CAR comprises an antigen-binding region.

The CAR may bind to human leukocyte antigen A2 (HLA-A2). In some embodiments, the CAR binds to tissue specific antigens involved in autoimmune diseases.

The antigen-binding region may comprise a light chain variable region (VL) and a heavy chain variable region (VH). The antigen-binding region may comprise a single-chain variable fragment (scFv). The cell may be substantially devoid of endogenous T-cell receptors (TCRs).

In one embodiment, the CAR comprises a cytoplasmic signaling domain of CD35.

The present disclosure also provides for methods of using the disclosed cells and compositions.

The present cells and compositions may be used in various therapeutic, prophylactic, diagnostic and other methods. The present cells and compositions and methods may be used in any situation in which immunosuppression is desired, e.g., transplant rejection or autoimmune disorders. The present cells and compositions and methods may be used to reduce complications associated with organ or tissue transplantation, reduce the likelihood of transplant rejection, prevent transplant rejection, treat transplant rejection, and induce immune tolerance. The present cells and composition and methods may be used to prevent and/or treat an autoimmune disorder. The present cells and composition and methods may be used to prevent and/or treat graft-versus-host disease. The compositions contain the disclosed cells.

Thus, one embodiment of the present disclosure is a method of inducing immunosuppression in a subject in need thereof comprising administering a therapeutically effective amount of the disclosed cells.

A further embodiment of the present disclosure is a method of inducing immunosuppression in a subject in need thereof comprising administering a therapeutically effective amount of a composition comprising the disclosed cells.

Another embodiment of the present disclosure is a method of inducing immune tolerance in a subject in need thereof comprising administering a therapeutically effective amount of the disclosed cells.

Yet a further embodiment of the present disclosure is a method of inducing immune tolerance in a subject in need thereof comprising administering a therapeutically effective amount of a composition comprising the disclosed cells.

Yet another embodiment of the present disclosure is a method of reducing complications associated with organ or tissue transplantation, reducing the likelihood of transplant rejection, preventing transplant rejection, or treating transplant rejection in a subject in need thereof comprising administering a therapeutically effective amount of the disclosed cells.

A further embodiment of the present disclosure is a method of reducing complications associated with organ or tissue transplantation, reducing the likelihood of transplant rejection, preventing transplant rejection, or treating transplant rejection in a subject in need thereof comprising administering a therapeutically effective amount of a composition comprising the disclosed cells.

One embodiment of the present disclosure is a method of preventing and/or treating an autoimmune disorder or disease in a subject in need thereof comprising administering a therapeutically effective amount of the disclosed cells.

A further embodiment of the present disclosure is a method of preventing and/or treating an autoimmune disorder or disease in a subject in need thereof comprising administering a therapeutically effective amount of a composition comprising the disclosed cells.

One embodiment of the present disclosure is a method of preventing and/or treating graft-versus-host disease in a subject in need thereof comprising administering a therapeutically effective amount of the disclosed cells.

A further embodiment of the present disclosure is a method of preventing and/or treating graft-versus-host disease in a subject in need thereof comprising administering a therapeutically effective amount of a composition comprising the disclosed cells.

In some embodiments, the compositions are pharmaceutical compositions.

In embodiments related to transplantation, the administration may be before, during and/or after the transplantation of the organ or tissue or cells.

In some embodiments, the organ or tissue being transplanted is from an allogenic donor. In some embodiment, the organ or tissue being transplanted is from xenogenic donor.

The present Treg cells may be used to suppress rejection responses to donor organs where the specific antigens are unknown.

### Definitions

The terms “subject,” “individual,” and “patient” are used interchangeably, and refer to a vertebrate, preferably a mammal such as a human. Mammals include, but are not limited to, human primates, non-human primates or murine, bovine, equine, canine or feline species. In the context of the present disclosure, the term “subject” also encompasses tissues and cells that can be cultured in vitro or ex vivo or manipulated in vivo. The term “subject” can be used interchangeably with the term “organism”.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Examples of polynucleotides include, but are not limited to, coding or non-coding regions of a gene or gene fragment, messenger RNA (mRNA), cDNA, recombinant polynucleotides, branched polynucleotides, plasmids, vectors, isolated DNA of any sequence, isolated RNA of any sequence, nucleic acid probes, and primers. One or more nucleotides within a polynucleotide can further be modified. The sequence of nucleotides may be interrupted by non-nucleotide components.

The term “genetically engineered” or “genetically modified” refers to cells being manipulated by genetic engineering, for example by genome editing. That is, the cells contain a heterologous sequence which does not naturally occur in said cells. The heterologous nucleic acid molecule may be integrated into the genome of the cells or may be present extra-chromosomally, e.g., in the form of plasmids. The term also includes embodiments of introducing genetically engineered, isolated CAR polypeptides into the cell.

The term “gRNA” or “guide RNA” or “sgRNA” or “single guide RNA” as used herein refers to the guide RNA sequences used to target specific genes for correction employing the CRISPR technique. Techniques of designing gRNAs and donor therapeutic polynucleotides for target specificity are well known in the art. For example, Doench, et al. 2014. Nature biotechnology 32 (12): 1262-7, Mohr, et al. 2016. FEBS Journal 3232-38, and Graham, et al. 2015. Genome Biol. 16:260. gRNA comprises or alternatively consists essentially of, or yet further consists of a fusion polynucleotide comprising CRISPR RNA (crRNA) and trans-activating CRISPR RNA (tracrRNA); or a polynucleotide comprising CRISPR RNA (crRNA) and trans-activating CRISPR RNA (tracrRNA). In some aspects, a gRNA is synthetic (Kelley, et al. 2016. J of Biotechnology 233:74-83). As used herein, a biological equivalent of a gRNA includes but is not limited to polynucleotides or targeting molecules that can guide a Cas9 or equivalent thereof to a specific polynucleotide sequence such as a specific region of a cell's genome.

As used herein, the terms “under the control”, “under transcriptional control”, “operably positioned”, and “operably linked” mean that a promoter is in a correct functional location and/or orientation in relation to a nucleic acid sequence, a DNA fragment, or a gene, to control transcriptional initiation and/or expression of that sequence, DNA fragment or gene.

The term “autologous” refers to any material derived from the same individual to whom it is later to be re-introduced into the same individual.

The term “allogeneic” refers to any material derived from a different animal of the same species as the individual to whom the material is introduced. Two or more individuals are said to be allogeneic to one another when the genes at one or more loci are not identical.

The terms “treat”, “treatment”, and the like refer to a means to slow down, relieve, ameliorate or alleviate at least one of the symptoms of the disease or disorder, or reverse the disease or disorder after its onset.

The terms “prevent”, “prevention”, and the like refer to acting prior to overt disease or disorder onset, to prevent the disease or disorder from developing or minimize the extent of the disease or disorder, or slow its course of development.

### Modified Regulatory T-cells (Tregs)

Natural regulatory T-cells (Tregs) are CD4⁺CD25⁺FOXP3⁺ T lymphocytes that control innate and adaptive immune responses. Natural Tregs also express low amounts of CD127, develop in the thymus, express GITR and CTLA-4. Tregs suppress effector T (Teff) cells from destroying their (self-) target, either through cell-cell contact by inhibiting T cell help and activation, through release of immunosuppressive cytokines such as IL-10 or TGF-β, through production of cytotoxic molecules such as granzyme B, through depleting IL-2 levels, or by changing the availability of specific nutrients in tissues.

As shown herein, Treg cells that were engineered such that they were deficient in or substantially devoid of a cell-surface marker or antigen, e.g., CD2, had increased functionality as compared CD2+ Treg cells and were both conversion-resistant and condition-resistant. Both Tregs and CAR Tregs which were engineered toto be deficient in or substantially devoid of a cell-surface marker or antigen, e.g., CD2, had these advantages.

Tregs can be genetically modified using recombinant techniques. Targeted or untargeted gene knockout methods can be used to engineer subject Tregs ex vivo prior to infusion into the subject. For example, the target DNA in the genome can be manipulated by deletion, insertion, and/or mutation using retroviral insertion, artificial chromosome techniques, gene insertion, random insertion with tissue specific promoters, gene targeting, transposable elements and/or any other method for introducing foreign DNA or producing modified DNA/modified nuclear DNA. Other modification techniques include deleting DNA sequences from a genome and/or altering nuclear DNA sequences. Nuclear DNA sequences, for example, may be altered by site-directed mutagenesis. Such methods generally use host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein. Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals. For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those which confer resistance to drugs, such as G418, hygromycin and methotrexate. Cells stably transfected with the introduced nucleic acid can be identified by drug selection (e.g., cells that have incorporated the selectable marker gene will survive, while the other cells die).

A CRISPR-Cas system can be used for precise editing of genomic nucleic acids (e.g., for creating null mutations). In such embodiments, the CRISPR guide RNA and/or the Cas enzyme (e.g., Cas9) may be expressed. Similar strategies may be used (e.g., designer zinc finger, transcription activator-like effectors (TALEs) or homing meganucleases). Such systems are well-known in the art (see, for example, U.S. Pat. No. 8,697,359; Sander and Joung (2014) Nat. Biotech. 32:347-355; Hale et al. (2009) Cell 139:945-956; Karginov and Hannon (2010) Mol. Cell 37:7; U.S. Pat. Publ. 2014/0087426 and 2012/0178169; Boch et al. (2011) Nat. Biotech. 29:135-136; Boch et al. (2009) Science 326:1509-1512; Moscou and Bogdanove (2009) Science 326:1501; Weber et al. (2011) PLOS One 6: e19722; Li et al. (2011) Nucl. Acids Res. 39:6315-6325; Zhang et al. (2011) Nat. Biotech. 29:149-153; Miller et al. (2011) Nat. Biotech. 29:143-148; Lin et al. (2014) Nucl. Acids Res. 42: e47). Such genetic strategies can use constitutive expression systems or inducible expression systems according to well-known methods in the art.

Disclosed herein is a CRISPR/Cas system for the removal of a cell-surface marker, e.g., CD2, from the Tregs. The CRISPR (Clustered Regularly Interspaced Short Palindromic Repeats) system exploits RNA-guided DNA-binding and sequence-specific cleavage of target DNA. The guide RNA/Cas combination confers site specificity to the nuclease. A single guide RNA (sgRNA) contains about 20 nucleotides that are complementary to a target genomic DNA sequence upstream of a genomic PAM (protospacer adjacent motifs) site (e.g., NGG) and a constant RNA scaffold region. The Cas (CRISPR-associated) protein binds to the sgRNA and the target DNA to which the sgRNA binds and introduces a double-strand break in a defined location upstream of the PAM site. Cas9 harbors two independent nuclease domains homologous to HNH and RuvC endonucleases, and by mutating either of the two domains, the Cas9 protein can be converted to a nickase that introduces single-strand breaks (Cong, et al. 2013 Science 339:819-823). It is specifically contemplated that the methods and compositions of the present disclosure can be used with the single- or double-strand-inducing version of Cas9, as well as with other RNA-guided DNA nucleases, such as other bacterial Cas9-like systems. The sequence-specific nuclease of the present methods and compositions described herein can be engineered, chimeric, or isolated from an organism. The nuclease can be introduced into the cell in form of a DNA, mRNA and protein.

In one embodiment, the methods of the present disclosure comprise using one or more sgRNAs to target and/or remove and/or inhibit CD2.

In some embodiments, the sgRNA to target and/or remove and/or inhibit CD2 has one of the following sequences set forth in Table 1.

TABLE 1
Sequence of single guide RNAs

| Guide | Guide | Targeting | Type of | | Where it |
|---|---|---|---|---|---|
| # | name | strand | sequence | Sequence | targets |
| 1 | Human | Coding | Reverse | TTACATGGAAAGCTCATCT | Exon1/ |
| | CD2 | | complemented | T (SEQ ID NO: 1) | Intron 1 |
| | guide 1 | | | | boundary |
| 2 | Human | Template | 5′ to 3′ | TTACGAATGCCTTGGAAAC | Exon 2 |
| | CD2 | | | C (SEQ ID NO: 2) | |
| | guide 2 | | | | |
| 3 | Human | Template | 5′ to 3′ | GCATCTGAAGACCGATGAT | Exon 2 |
| | CD2 | | | C (SEQ ID NO: 3) | |
| | guide 3 | | | | |
| 4 | Human | Coding | Reverse | CTTGATACAGGTTTAATTCG | Exon 3 |
| | CD2 | | complemented | (SEQ ID NO: 4) | |
| | guide 4 | | | | |
| 5 | Human | Coding | Reverse | CACGCACCTGGACAGCTGA | Exon 3/ |
| | CD2 | | complemented | C (SEQ ID NO: 5) | Intron 4 |
| | guide 5 | | | | boundary |

In one embodiment, the DNA digesting agent can be a site-specific nuclease. In another embodiment, the site-specific nuclease may be a Cas-family nuclease. In a more specific embodiment, the Cas nuclease may be a Cas9 nuclease.

In one embodiment, Cas protein may be a functional derivative of a naturally occurring Cas protein.

In some embodiments, the nucleotide sequence encoding the Cas (e.g., Cas9) nuclease is modified to alter the activity of the protein. In some embodiments, the Cas (e.g., Cas9) nuclease is a catalytically inactive Cas (e.g., Cas9) (or a catalytically deactivated/defective Cas9 or dCas9). In one embodiment, dCas (e.g., dCas9) is a Cas protein (e.g., Cas9) that lacks endonuclease activity due to point mutations at one or both endonuclease catalytic sites (RuvC and HNH) of wild type Cas (e.g., Cas9). For example, dCas9 contains mutations of catalytically active residues (D10 and H840) and does not have nuclease activity. In some cases, the dCas has a reduced ability to cleave both the complementary and the non-complementary strands of the target DNA. In some cases, the dCas9 harbors both D10A and H840A mutations of the amino acid sequence of S. pyogenes Cas9. In some embodiments when a dCas9 has reduced catalytic activity (e.g., when a Cas9 protein has a D10, G12, G17, E762, H840, N854, N863, H982, H983, A984, D986, and/or a A987 mutation, e.g., D10A, G12A, G17A, E762A, H840A, N854A, N863A, H982A, H983A, A984A, and/or D986A), the Cas protein can still bind to target DNA in a site-specific manner, because it is still guided to a target polynucleotide sequence by a DNA-targeting sequence of the subject polynucleotide (e.g., gRNA), as long as it retains the ability to interact with the Cas-binding sequence of the subject polynucleotide (e.g., gRNA).

The present methods and systems may use CRISPR deletion (CRISPRd). CRISPRd capitalizes on the tendency of DNA repair strategies to default towards NHEJ and does not require a donor template to repair the cleaved strand. Instead, Cas creates a DSB in the gene harboring a mutation first, then NHEJ occurs, and insertions and/or deletions (INDELs) are introduced that corrupt the sequence, thus either preventing the gene from being expressed or proper protein folding from occurring. This strategy may be particularly applicable for dominant conditions, in which case knocking out the mutated, dominant allele and leaving the wild type allele intact may be sufficient to restore the phenotype to wild type.

In addition to well characterized CRISPR-Cas system, a new CRISPR enzyme, called Cpf1 (Cas protein 1 of PreFran subtype) may be used in the present methods and systems (Zetsche et al. 2015. Cell). Cpf1 is a single RNA-guided endonuclease that lacks tracrRNA, and utilizes a T-rich protospacer-adjacent motif. The authors demonstrated that Cpf1 mediates strong DNA interference with characteristics distinct from those of Cas9. Thus, in one embodiment of the present invention, CRISPR-Cpf1 system can be used to cleave a desired region within the targeted gene.

Guide RNA(s) or single guide RNA(s) used in the methods of the present disclosure can be designed so that they direct binding of the Cas-gRNA complexes to pre-determined cleavage sites in a genome. In one embodiment, the cleavage sites may be chosen so as to release a fragment or sequence that contains a region of a frame shift mutation. In further embodiment, the cleavage sites may be chosen so as to release a fragment or sequence that contains an extra chromosome.

For Cas family enzyme (such as Cas9) to successfully bind to DNA, the target sequence in the genomic DNA can be complementary to the gRNA sequence and may be immediately followed by the correct protospacer adjacent motif or “PAM” sequence. “Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule, which can form hydrogen bonds (e.g., Watson-Crick base pairing) with a second nucleic acid sequence. Full complementarity is not necessarily required, provided there is sufficient complementarity to cause hybridization and promote formation of a CRISPR complex. A target sequence may comprise any polynucleotide, such as DNA polynucleotides. The Cas9 protein can tolerate mismatches distal from the PAM. The PAM sequence varies by the species of the bacteria from which Cas9 was derived. The most widely used CRISPR system is derived from S. pyogenes and the PAM sequence is NGG located on the immediate 3′ end of the sgRNA recognition sequence. The PAM sequences of CRISPR systems from exemplary bacterial species include: Streptococcus pyogenes (NGG), Neisseria meningitidis (NNNNGATT), Streptococcus thermophilus (NNAGAA) and Treponema denticola (NAAAAC).

gRNA(s) used in the present disclosure can be between about 5 and 100 nucleotides long, or longer (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 60, 61, 62, 63, 63, 64, 65, 66, 67, 68, 69, 70, 71, 72, 73, 74, 75, 76, 77, 78, 79, 80, 81, 82, 83, 84, 85, 86, 87, 88, 89, 90, 91 92, 93, 94, 95, 96, 97, 98, 99, or 100 nucleotides in length, or longer). In one embodiment, gRNA(s) can be between about 15 and about 30 nucleotides in length (e.g., about 15-29, 15-26, 15-25; 16-30, 16-29, 16-26, 16-25; or about 18-30, 18-29, 18-26, or 18-25 nucleotides in length).

To facilitate gRNA design, many computational tools have been developed (See Prykhozhij et al. 2015 PLOS ONE 10 (3): Zhu et al. 2014 PLOS ONE 9 (9); Xiao et al. 2014 Bioinformatics. January 21 (2014)); Heigwer et al. 2014 Nat Methods 11 (2): 122-123). Methods and tools for guide RNA design are discussed by Zhu 2015 Frontiers in Biology 10 (4): 289-296, which is incorporated by reference herein. Additionally, there is a publicly available software tool that can be used to facilitate the design of gRNA(s).

The RNA-guided nuclease can be introduced into the cell in the form of a protein or in the form of a nucleic acid encoding the sequence-specific nuclease, such as an mRNA or a cDNA. The gRNA can be introduced into the cell as an RNA or as a DNA encoding the gRNA. Nucleic acids can be delivered as part of a larger construct, such as a plasmid or viral vector, or directly, e.g., by electroporation, lipid vesicles, viral transporters, microinjection, and biolistics. Similarly, the construct containing the one or more transgenes can be delivered by any method appropriate for introducing nucleic acids into a cell.

In embodiments in which both the RNA-guided endonuclease and the guide RNA are introduced into the cell as DNA molecules, each can be part of a separate molecule (e.g., one vector containing endonuclease coding sequence and a second vector containing guide RNA coding sequence) or both can be part of the same molecule (e.g., one vector containing coding (and regulatory) sequence for both the endonuclease and the guide RNA).

In some embodiments, the RNA-guided endonuclease and gRNA are introduced into the cell in the form of a ribonucleoprotein complex comprising the endonuclease complexed to least one gRNA. Preparation of such RNP complexes are known in the art or can be obtained commercially.

The gRNA/RNA guided endonuclease can be delivered to the subject or cell using one or more viruses including recombinant adeno-associated viral (AAV) vectors (e.g., 1, 2, 3, 4, 5, 6, 7, 8, 9, or more AAV vectors). One or more gRNAs (e.g., sgRNAs) can be packaged into single (one) recombinant AAV vector. An RNA-guided endonuclease can be packaged into the same, or alternatively separate recombinant AAV vectors.

In these embodiments, a variety of known viral constructs may be used to deliver the sgRNA(s) and endonucleases to the targeted cells and/or a subject. Nonlimiting examples of such recombinant viruses include recombinant adeno-associated virus (AAV), recombinant adenoviruses, recombinant lentiviruses, recombinant retroviruses, recombinant poxviruses, and other known viruses in the art, as well as plasmids, cosmids, and phages. Options for gene delivery viral constructs are well known.

Additionally, delivery vehicles such as nanoparticle- and lipid-based mRNA or protein delivery systems can be used as an alternative to AAV vectors. Further examples of alternative delivery vehicles include lentiviral vectors, ribonucleoprotein (RNP) complexes, lipid-based delivery system, gene gun, hydrodynamic, electroporation or nucleofection microinjection, and biolistics.

Also as described herein and in some embodiments, Tregs are administered to a subject. Thus, the Tregs will have an immunocompatibility relationship to the subject and any such relationship is contemplated for use according to the present methods. For example, the Tregs can be syngeneic. The term “syngeneic” can refer to the state of deriving from, originating in, or being members of the same species that are genetically identical, particularly with respect to antigens or immunological reactions. Thus, the Tregs may be from a donor to a recipient who is genetically identical to the donor or is sufficiently immunologically compatible as to allow for transplantation without an undesired adverse immunogenic response. The Tregs may be autologous if the transferred cells are obtained from and administered to the same subject. The Tregs may be the subject's own cells which are harvested from, modified, and reinfused to the subject. The Tregs may be allogeneic where the cells are from a different animal/individual of the same species as the individual to whom the cells are introduced. Two or more individuals are said to be allogeneic to one another when the genes at one or more loci are not identical.

In addition, Tregs can be obtained from a single source or a plurality of sources (e.g., a single subject or a plurality of subjects). A plurality refers to at least two (e.g., more than one).

### Immune Cells Comprising Chimeric Antigen Receptor(s) (CAR(s))

Chimeric antigen receptor (CAR) T cells are widely used to recognize antigens on cells with both high affinity and specificity and without the requirement for accessory recognition molecules, such as HLA antigens to “present” peptides. The T cell receptor of a CAR T cells is “swapped” with an antigen-binding heavy and light chains, thereby obviating the need for HLA accessory molecules.

In particular aspects, the immune cells are T cells or Treg cells that express a CAR.

A CAR is an artificially constructed hybrid protein or polypeptide typically containing an extracellular antigen binding domain and a transmembrane domain. The recombinant CAR may or may not be fused to signaling domains leading to activation of the T cell upon binding of the CAR to its target antigen. Characteristics of CARs include their ability to redirect T-cell specificity and reactivity toward a selected target in a non-MHC-restricted manner, exploiting the antigen-binding properties of monoclonal antibodies. The non-MHC-restricted antigen recognition gives T cells expressing CARs the ability to recognize antigen independent of antigen processing. Moreover, when expressed in T-cells, CARs advantageously do not dimerize with endogenous T cell receptor (TCR) alpha and beta chains.

In one embodiment, the stimulatory molecule is the zeta chain associated with the T cell receptor complex. In one aspect, the cytoplasmic signaling domain further comprises one or more functional signaling domains derived from at least one costimulatory molecule as defined below. The costimulatory molecule may also be 4-1BB (i.e., CD137), CD27 and/or CD28 or fragments of those molecules. In one embodiment, the CAR comprises a chimeric fusion protein comprising an extracellular antigen recognition domain, a transmembrane domain and an intracellular signaling domain comprising a functional signaling domain derived from a stimulatory molecule. In one embodiment, the CAR comprises a chimeric fusion protein comprising an extracellular antigen recognition domain, a transmembrane domain and an intracellular signaling domain comprising a functional signaling domain derived from a co-stimulatory molecule and a functional signaling domain derived from a stimulatory molecule. Alternatively, the CAR may comprise a chimeric fusion protein comprising an extracellular antigen recognition domain, a transmembrane domain and an intracellular signaling domain comprising two functional signaling domains derived from one or more co-stimulatory molecule(s) and a functional signaling domain derived from a stimulatory molecule. The CAR can also comprise a chimeric fusion protein comprising an extracellular antigen recognition domain, a transmembrane domain and an intracellular signaling domain comprising at least two functional signaling domains derived from one or more co-stimulatory molecule(s) and a functional signaling domain derived from a stimulatory molecule. The antigen recognition moiety of the CAR encoded by the nucleic acid sequence can contain any lineage specific, antigen-binding antibody fragment. The antibody fragment can comprise one or more CDRs, the variable region (or portions thereof), the constant region (or portions thereof), or combinations of any of the foregoing.

The term “signaling domain” refers to the functional portion of a protein which acts by transmitting information within the cell to regulate cellular activity via defined signaling pathways by generating second messengers or functioning as effectors by responding to such messengers.

The term “zeta” or alternatively “zeta chain”, “CD3-zeta” or “TCR-zeta” is defined as the protein provided as GenBank accession numbers NP_932170, NP_000725, or XP_011508447; or the equivalent residues from a non-human species, e.g., mouse, rodent, monkey, ape and the like, and a “zeta stimulatory domain” or alternatively a “CD3-zeta stimulatory domain” or a “TCR-zeta stimulatory domain” is defined as the amino acid residues from the cytoplasmic domain of the zeta chain that are sufficient to functionally transmit an initial signal necessary for T cell activation.

The phrases “have antigen specificity” and “elicit antigen-specific response” as used herein means that the CAR can specifically bind to and immunologically recognize an antigen, such that binding of the CAR to the antigen elicits an immune response.

The extracellular antigen binding domain may be any protein or portion thereof that binds to a target protein, e.g., a receptor or ligand-binding portion thereof; a ligand of a receptor (e.g., a cytokine); or an antibody or antigen-binding portion of an antibody, e.g., a single-chain antibody (scFv).

In certain embodiments, a CAR comprises a transmembrane domain selected from the group consisting of a CD4 transmembrane domain, a CD8 transmembrane domain, and a CD28 transmembrane domain.

In certain embodiments, the intracellular signaling domain comprises a primary signaling domain, e.g., a T cell receptor zeta chain or primary signaling domain therefrom. In certain embodiments, the intracellular signaling domain further comprises one or more co-stimulatory domains. Illustrative examples of co-stimulatory domains that may be used in the CARs may include, but are not limited to CD27, CD28, CD137 (4-1BB), OX-40, or combinations thereof.

The CAR may be a first-generation, second-generation, or third-generation CAR.

In particular embodiments, the CAR is encoded by an expression vector. The vector may be bicistronic, in particular embodiments. In some embodiments, more than one CAR is expressed by the immune cell. In particular embodiments where more than one CAR is to be expressed by the immune cell, the two or more CAR expression constructs may or may not be on the same vector. When present on the same vector, the first CAR coding sequence may be configured 5′ or 3′ to the second CAR coding sequence. The expression of the first CAR and second or subsequent CAR receptor may be under the direction of the same or different regulatory sequences.

### Transplantation

The present cells and compositions and methods may be used to reduce complications associated with organ or tissue transplantation, reduce the likelihood of transplant rejection, prevent transplant rejection, treat transplant rejection, and induce immune tolerance. The present cells and compositions and methods can be used in conjunction with transplantation of any organ or any tissue that is suitable for transplantation. Non-limiting exemplary organs include heart, kidney, lung, liver, pancreas, intestine, and thymus. Non-limiting exemplary tissues include bone, tendon, cornea, skin, heart valve, vein, and bone marrow. The method may comprise administering the present cells and compositions to the subject before, during and/or after transplantation.

The present disclosure provides for a method of inducing immune tolerance, or treating or preventing rejection, for transplantation in a subject to a graft obtained from an allogenic donor mammal. The method may comprise administering the present cells and compositions to the subject before, during and/or after transplantation.

The term “allogeneic” refers to any material derived from a different animal of the same species as the individual to whom the material is introduced. An “allogeneic transplantation” refers to transfer of cells, tissues or organs from a donor to a recipient where the recipient and the donor are the same species.

The present disclosure provides for a method of inducing immune tolerance, or treating or preventing rejection, for xenotransplantation in a subject to a graft obtained from a donor mammal. The method may comprise administering the present cells and compositions to the subject before, during and/or after transplantation.

“Xenogeneic” refers to deriving from, originating in, or being members of different species, e.g., human and swine, human and chimpanzee, human and rodent. A “xenogeneic transplantation” or “xenotransplantation” refers to transfer of cells, tissues or organs from a donor to a recipient where the recipient is a species different from that of the donor.

The second species may be swine, such as a miniature swine. “Miniature swine”, as used herein, refers to completely or partially inbred miniature swine.

The first species may be primate, such as non-human primate or human.

“Graft”, as used herein, refers to a body part, organ, tissue, cells, or portions thereof.

Thus, the graft may comprise cells, a tissue or an organ. In one embodiment, the graft comprises hematopoietic stem cells. In another embodiment, the graft comprises bone marrow. In yet another embodiment, the graft comprises a heart, a kidney, a liver, a pancreas, a lung, an intestine, skin, a small bowel, a trachea, a cornea, or combinations thereof.

“Tolerance”, as used herein, refers to the inhibition or decrease of a graft recipient's ability to mount an immune response, e.g., to a donor antigen, which would otherwise occur, e.g., in response to the introduction of a non self MHC antigen into the recipient. Tolerance can involve humoral, cellular, or both humoral and cellular responses. The concept of tolerance includes both complete and partial tolerance. In other words, as used herein, tolerance include any degree of inhibition of a graft recipient's ability to mount an immune response, e.g., to a donor antigen.

Methods of the present disclosure can be used to confer tolerance to xenogeneic grafts, e.g., wherein the graft donor is a nonhuman animal, e.g., a swine, e.g., a miniature swine, and the graft recipient is a primate, e.g., a human.

The donor of the xenograft and the individual that supplies the tolerance-inducing thymic tissue may be the same individual or may be as closely related as possible. For example, it is preferable to derive a xenograft from a colony of donors that is highly or completely inbred.

The donor may be a non-human mammalian species, such as a swine or a non-human primate. Non-limiting examples of the donor include a swine, rodent, non-human primate, cow, goat, and horse.

The recipient may be a primate, such as non-human primate (e.g., a baboon, or cynomolgus monkey) or human. In one embodiment, the recipient is human.

In certain embodiments, the donor and recipient are of different species. For example, the donor is a non-human animal, e.g., a miniature swine, and the recipient is a human.

Also encompassed by the present disclosure are methods of transplanting a graft from such a donor animal into a recipient (e.g., human).

Cells, tissues, organs or body fluids of the present donor animal may be used for transplantation (e.g., xenotransplantation). The graft harvested from the donor for transplantation may include, but are not limited to, a heart, a kidney, a liver, a pancreas, a lung transplant, an intestine, skin, thyroid, bone marrow, small bowel, a trachea, a cornea, a limb, a bone, an endocrine gland, blood vessels, connective tissue, progenitor stem cells, blood cells, hematopoietic cells, Islets of Langerhans, brain cells and cells from endocrine and other organs, bodily fluids, and combinations thereof.

The cell can be any type of cell. In certain embodiments, the cell is a hematopoietic cell (e.g., a hematopoietic stem cell, lymphocyte, a myeloid cell), a pancreatic cell (e.g., a beta-islet cell), a kidney cell, a heart cell, or a liver cell.

Bone marrow cells, or hematopoietic stem cells (e.g., a fetal liver suspension or mobilized peripheral blood stem cells) of the donor animal may be injected into the recipient.

In some embodiments, donor stromal tissue is administered. It may be obtained from fetal liver, thymus, and/or fetal spleen, may be implanted into the recipient, e.g., in the kidney capsule. Thymic tissue can be prepared for transplantation by implantation under the autologous kidney capsule for revascularization. Stem cell engraftment and hematopoiesis across disparate species barriers may be enhanced by providing a hematopoictic stromal environment from the donor species. The stromal matrix supplies species-specific factors that are required for interactions between hematopoietic cells and their stromal environment, such as hematopoictic growth factors, adhesion molecules, and their ligands.

As liver is the major site of hematopoiesis in the fetus, fetal liver can also serve as an alternative to bone marrow as a source of hematopoietic stem cells. Each organ includes an organ specific stromal matrix that can support differentiation of the respective undifferentiated stem cells implanted into the host. As an alternative or an adjunct to implantation, fetal liver cells can be administered in fluid suspension.

Bone marrow cells, or another source of hematopoietic stem cells, e.g., a fetal liver suspension, of the donor can be injected into the recipient. Donor bone marrow cells home to appropriate sites of the recipient and grow contiguously with remaining host cells and proliferate, forming a chimeric lymphohematopoietic population. By this process, newly forming B cells (and the antibodies they produce) are exposed to donor antigens, so that the transplant will be recognized as self. Tolerance to the donor is also observed at the T cell level in animals in which hematopoietic stem cell, e.g., bone marrow cells, engraftment has been achieved. The use of xenogeneic donors allows the possibility of using bone marrow cells and organs from the same animal, or from genetically matched animals.

### Autoimmune Diseases and Disorders

The present cells/compositions and methods may have in vitro and in vivo therapeutic, prophylactic, and/or diagnostic utilities.

The present cells and compositions and methods may be used to treat or prevent an autoimmune disease or disorder.

The autoimmune disease or disorder may be associated with or caused by the presence of an autoantibody.

The autoimmune disorder may be systemic lupus erythematosus (SLE), CREST syndrome (calcinosis, Raynaud's syndrome, esophageal dysmotility, sclerodactyl, and telangiectasia), opsoclonus, inflammatory myopathy (e.g., polymyositis, dermatomyositis, and inclusion-body myositis), systemic scleroderma, primary biliary cirrhosis, celiac disease (e.g., gluten sensitive enteropathy), dermatitis herpetiformis, Miller-Fisher Syndrome, acute motor axonal neuropathy (AMAN), multifocal motor neuropathy with conduction block, autoimmune hepatitis, antiphospholipid syndrome, Wegener's granulomatosis, microscopic polyangiitis, Churg-Strauss syndrome, rheumatoid arthritis, chronic autoimmune hepatitis, scleromyositis, myasthenia gravis, Lambert-Eaton myasthenic syndrome, Hashimoto's thyroiditis, Graves' disease, Paraneoplastic cerebellar degeneration, Stiff person syndrome, limbic encephalitis, Isaacs Syndrome, Sydenham's chorea, pediatric autoimmune neuropsychiatric disease associated with Streptococcus (PANDAS), encephalitis, diabetes mellitus type 1, and/or Neuromyelitis optica.

The autoimmune disorder may be pernicious anemia, Addison's disease, psoriasis, inflammatory bowel disease, psoriatic arthritis, Sjögren's syndrome, lupus erythematosus (e.g., discoid lupus erythematosus, drug-induced lupus erythematosus, and neonatal lupus erythematosus), multiple sclerosis, and/or reactive arthritis.

The autoimmune disorder may be polymyositis, dermatomyositis, multiple endocrine failure, Schmidt's syndrome, autoimmune uveitis, adrenalitis, thyroiditis, autoimmune thyroid disease, gastric atrophy, chronic hepatitis, lupoid hepatitis, atherosclerosis, presenile dementia, demyelinating diseases, subacute cutaneous lupus erythematosus, hypoparathyroidism, Dressler's syndrome, autoimmune thrombocytopenia, idiopathic thrombocytopenia purpura, hemolytic anemia, pemphigus vulgaris, pemphigus, alopecia arcata, pemphigoid, scleroderma, progressive systemic sclerosis, adult onset diabetes mellitus (e.g., type II diabetes), male and female autoimmune infertility, ankylosing spondolytis, ulcerative colitis, Crohn's disease, mixed connective tissue disease, polyarteritis nedosa, systemic necrotizing vasculitis, juvenile onset rheumatoid arthritis, glomerulonephritis, atopic dermatitis, atopic rhinitis, Goodpasture's syndrome, Chagas' disease, sarcoidosis, rheumatic fever, asthma, recurrent abortion, anti-phospholipid syndrome, farmer's lung, erythema multiforme, post cardiotomy syndrome, Cushing's syndrome, autoimmune chronic active hepatitis, bird-fancier's lung, allergic disease, allergic encephalomyelitis, toxic epidermal necrolysis, alopecia, Alport's syndrome, alveolitis, allergic alveolitis, fibrosing alveolitis, interstitial lung disease, erythema nodosum, pyoderma gangrenosum, transfusion reaction, leprosy, malaria, leishmaniasis, trypanosomiasis, Takayasu's arteritis, polymyalgia rheumatica, temporal arteritis, schistosomiasis, giant cell arteritis, ascariasis, aspergillosis, Sampter's syndrome, eczema, lymphomatoid granulomatosis, Behcet's disease, Caplan's syndrome, Kawasaki's disease, dengue, endocarditis, endomyocardial fibrosis, endophthalmitis, erythema elevatum et diutinum, erythroblastosis fetalis, cosinophilic faciitis, Shulman's syndrome, Felty's syndrome, filariasis, cyclitis, chronic cyclitis, heterochronic cyclitis, Fuch's cyclitis, IgA nephropathy, Henoch-Schonlein purpura, graft versus host disease, transplantation rejection, human immunodeficiency virus infection, echovirus infection, cardiomyopathy, Alzheimer's disease, parvovirus infection, rubella virus infection, post vaccination syndromes, congenital rubella infection, Hodgkin's and non-Hodgkin's lymphoma, renal cell carcinoma, multiple myeloma, Eaton-Lambert syndrome, relapsing polychondritis, malignant melanoma, cryoglobulinemia, Waldenstrom's macroglobulemia, Epstein-Barr virus infection, mumps, Evan's syndrome, and/or autoimmune gonadal failure.

### Pharmaceutical Compositions

The present disclosure provides compositions, including pharmaceutical compositions, comprising the present cells.

The phrase “pharmaceutically acceptable” refers to molecular entities and compositions that are physiologically tolerable and do not typically produce an allergic or similar untoward reaction, such as gastric upset, dizziness and the like, when administered to a human, and approved by a regulatory agency of the Federal or a state government or listed in the U.S. Pharmacopeia or other generally recognized pharmacopeia for use in animals, and more particularly in humans. “Carrier” refers to a diluent, adjuvant, excipient, or vehicle with which the therapeutic is administered. Such pharmaceutical carriers can be sterile liquids, such as saline solutions in water and oils, including those of petroleum, animal, vegetable, or synthetic origin, such as peanut oil, soybean oil, mineral oil, sesame oil, and the like. A saline solution is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents.

The cells or pharmaceutical compositions may be administered by any route, including, without limitation, oral, transdermal, ocular, intraperitoneal, intravenous, ICV, intracisternal injection or infusion, subcutaneous, implant, sublingual, subcutaneous, intramuscular, intravenous, rectal, mucosal, ophthalmic, intrathecal, intra-articular, intra-arterial, sub-arachinoid, bronchial and lymphatic administration. The present composition may be administered parenterally or systemically.

Intravenous forms include, but are not limited to, bolus and drip injections. Examples of intravenous dosage forms include, but are not limited to, Water for Injection USP; aqueous vehicles including, but not limited to, Sodium Chloride Injection, Ringer's Injection, Dextrose Injection, Dextrose and Sodium Chloride Injection, and Lactated Ringer's Injection; water-miscible vehicles including, but not limited to, ethyl alcohol, polyethylene glycol and polypropylene glycol; and non-aqueous vehicles including, but not limited to, corn oil, cottonseed oil, peanut oil, sesame oil, ethyl oleate, isopropyl myristate and benzyl benzoate.

The present composition may be administered as a single dose, or as two or more doses (which may or may not contain the same amount of the desired molecule) over time, or as a continuous infusion via implantation device or catheter. The pharmaceutical composition can be prepared in single unit dosage forms.

Appropriate frequency of administration can be determined by one of skill in the art and can be administered once or several times per day (e.g., twice, three, four or five times daily). The compositions of the invention may also be administered once each day or once every other day. The compositions may also be given twice weekly, weekly, monthly, or semi-annually. In the case of acute administration, treatment is typically carried out for periods of hours or days, while chronic treatment can be carried out for weeks, months, or even years. U.S. Pat. No. 8,501,686.

Administration of the compositions can be carried out using any of several standard methods including, but not limited to, continuous infusion, bolus injection, intermittent infusion, or combinations of these methods. For example, one mode of administration that can be used involves continuous intravenous infusion. The infusion of the compositions of the invention can, if desired, be preceded by a bolus injection.

As used herein, the term “therapeutically effective amount” is an amount sufficient to treat a specified disorder or disease or alternatively to obtain a pharmacological response treating a disorder or disease.

Methods of determining the most effective means and dosage of administration can vary with the composition used for therapy, the purpose of the therapy, the target cell being treated, and the subject or patient being treated. Single or multiple administrations can be carried out with the dose level and pattern being selected by the treating physician. The specific dose level for any particular subject depends upon a variety of factors including the age, body weight, general health, sex, diet, time of administration, route of administration, and rate of excretion, and the severity of the particular disease undergoing therapy.

The disclosed Tregs may be administered at 0.1×10⁶, 0.2×10⁶, 0.3×10⁶, 0.4×10⁶, 0.5×10⁶, 0.6×10⁶, 0.7×10⁶, 0.8×10⁶, 0.9×10⁶, 1.0×10⁶, 5.0×10⁶, 1.0×10⁷, 5.0×10⁷, 1.0×10⁸, 5.0×10⁸, or more, or any range in between or any value in between, cells per kilogram of subject body weight. The number of cells administered may be adjusted. Generally, 1×10⁵ to about 1×10⁹ cells/kg of body weight, from about 1×10⁶ to about 1×10⁸ cells/kg of body weight, or about 1×10⁷ cells/kg of body weight, or more cells, as necessary, may be administered.

Different dosage regimens may be used. In some embodiments, a daily dosage, such as any of the exemplary dosages described above, is administered once, twice, three times, or four times a day for at least three, four, five, six, seven, eight, nine, or ten days. Depending on the stage and severity of the cancer, a shorter treatment time (e.g., up to five days) may be employed along with a high dosage, or a longer treatment time (e.g., ten or more days, or weeks, or a month, or longer) may be employed along with a low dosage. In some embodiments, a once- or twice-daily dosage is administered every other day.

Administration can be accomplished using methods generally known in the art. The cells/composition may be administered to the desired site by direct injection, or by any other means used in the art including, but are not limited to, intravascular, intracerebral, parenteral, intraperitoneal, intravenous, epidural, intraspinal, intrasternal, intra-articular, intra-synovial, intrathecal, intra-arterial, intracardiac, or intramuscular administration. For example, subjects of interest may be administered with the cells/composition by various routes. Such routes include, but are not limited to, intravenous administration, subcutaneous administration, administration to a specific tissue (e.g., focal transplantation), injection into the femur bone marrow cavity, injection into the spleen, administration under the renal capsule of fetal liver, and the like. Cells may be administered in one infusion, or through successive infusions over a defined time period sufficient to generate a desired effect.

Administration of a therapeutically active amount of the present cells and composition may be defined as an amount effective, at dosages and for periods of time necessary, to achieve the desired result. For example, a therapeutically active amount of the present cells/composition may vary according to factors such as the disease state, age, sex, and weight of the individual, and the ability of peptide to elicit a desired response in the individual. Dosage regimens can be adjusted to provide the optimum therapeutic response. For example, several divided doses can be administered daily or the dose can be proportionally reduced as indicated by the exigencies of the therapeutic situation.

### Kits

The present disclosure provides for a kit for producing the disclosed conversion-resistant/condition-resistant CAR Treg cells or Treg cells. In some embodiments, a kit described herein comprises a gRNA having a sequence of selected from the group SEQ ID NOs: 1-5 and combinations thereof. In some embodiments, the kit can further comprise reagents of CRISPR-based systems, including a Cas protein. The kit can further comprise instructions.

The present disclosure also provides for a kit using the disclosed cells and compositions for the treatment or prevention of an autoimmune disorder. The present disclosure also provides for a kit using the disclosed cells and compositions for inducing immune tolerance or treating or preventing transplant rejection.

Kits according to the present disclosure include package(s) (e.g., vessels) comprising the present cell or compositions. The cells may be present in the pharmaceutical compositions as described herein. The cells or compositions may be present in unit dosage forms.

Examples of pharmaceutical packaging materials include, but are not limited to, bottles, tubes, inhalers, pumps, bags, vials, containers, syringes, bottles, and any packaging material suitable for a selected formulation and intended mode of administration and treatment.

Kits can contain instructions for administering the present cells or compositions to a patient. Kits also can comprise instructions for uses of the present cells or compositions. Kits also can contain labeling or product inserts for the cells/compositions. The kits also can include buffers for preparing solutions for conducting the methods.

### EXAMPLES

### Example 1—Materials and Methods for Examples 2-4

### Guide RNA

The guide RNA sequences set forth in Table 1 were used. Guides #4 and #5 with SEQ ID NOs: 4 and 5, respectively, showed the highest efficiency in removing CD2 molecules from T cells. Guide #5 was used in the in vitro and in vivo experiments (Examples 3 and 4).

### Source of Treg Cells

Human peripheral blood buffy coats were requested from NY Blood Center. Upon ficol centrifugation, mononuclear buffy coat was isolated. CD25+ cell were enriched using magnetic cell sorting. Tregs (CD4+ CD25+ CD127−) were FACS-sorted from CD25-enriched cells. Sorted Tregs were activated with CD3/28 beads.

Introduction of the gRNA/Cas9 to the Treg Cells

After 2-3 days of activation, cells were washed with PBS, counted and mixed with the ribonucleoprotein (RNP) complex containing the Cas9 enzyme and crRNA: tracrRNA guide complex. The mixture was electroporated using the Neon machine (Voltage: 1600V, Width: 10 ms, pulse: 3). Electroporated cells were immediately transferred to the culture media. HLA-A2 CAR lentivirus was added to the flat-bottom 96-well plate wells that were treated with retronectin (MOI: 20). After 1 h of centrifugation at 2000 g, electroporated and non-electroporated Treg cells were added to the wells either containing or not containing the virus (1-3×10⁵ cells per well). X-Vivo15 media containing recombinant transferrin, 10% human serum and 500 U/ml IL-2 was used for Treg culture. After 10-14 days of expansion, Tregs were used for in vitro and in vivo assays.

### In Vitro Testing

Thawed CD25-depleted PBMCs were stained with CFSE and used as effector cells in Treg suppression assay (150K cells per well). Tregs were added at different ratios of Treg: Teff (1:1, 1:2, 1:4, . . . , 1:128, 0:1). HLA-A2+ fetal liver HSC-derived dendritic cells were used as stimulators in the assay (50K-60k per well).

After 6 days, cells were stained and analyzed with flow cytometry. This formula was used to measure the % of Treg suppression: “(% proliferating effectors without Tregs−% proliferating effectors with Tregs)/% proliferating effectors without Tregs))*100)”.

Introduction of the Cells into Mice and Testing In Vivo:

HLA-A2 Tg NSG mice were irradiated (100cGy). 5 million thawed CD25-depleted PBMCs were injected i.v to HLA-A2 Tg NSG mice in order to induce GVHD. 800K Tregs from the 4 Treg groups were injected to prevent GVHD.
Group 1: No Tregs
Group 2: No CAR/CD2+ Tregs
Group 3: No CAR/CD2-KO Tregs
Group 4: HLA-A2 CAR/CD2+ Tregs
Group 5: HLA-A2 CAR/CD2-KO Tregs

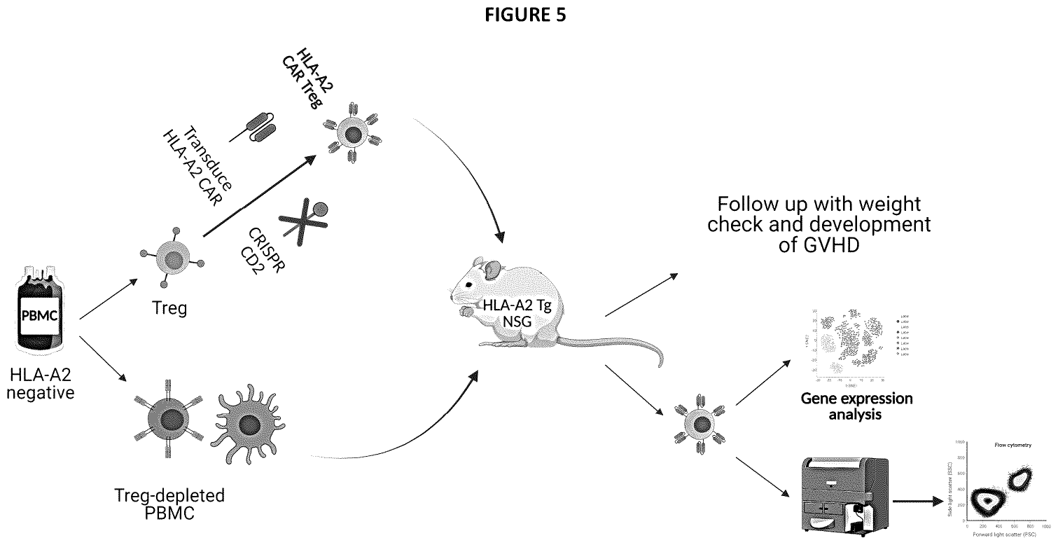

Mice were followed by measuring weight and scoring for GVHD. Also, immune cells were analyzed in peripheral blood using flow cytometry. See FIG. 5.

### Example 2—CRISPR-Removal of CD2 Molecule for CD4 T Cells Resulted in Stable Cells with the Ability to Expand

The five guide RNA with sequences SEQ ID NOs: 1-5 were used in a CRISPR-Cas9 system to remove CD2 from Treg cells.

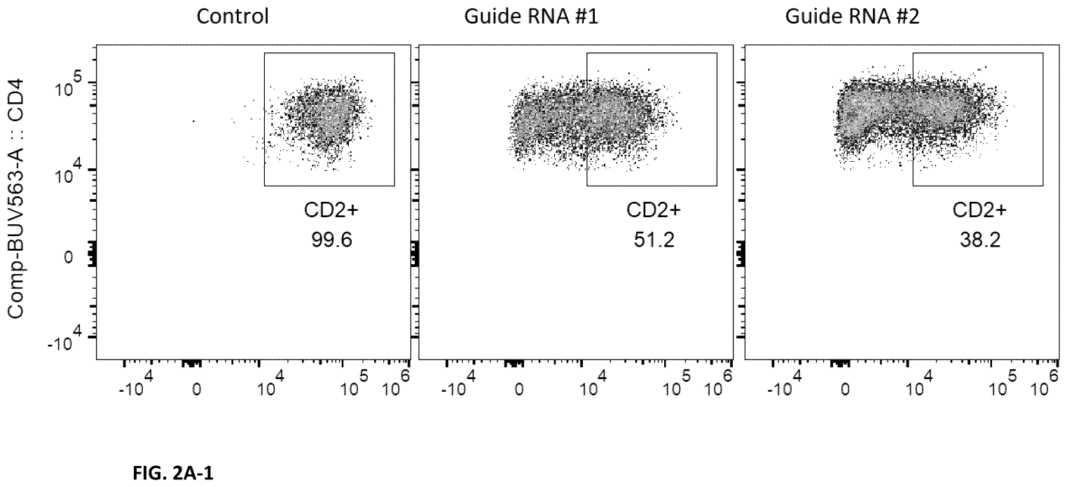

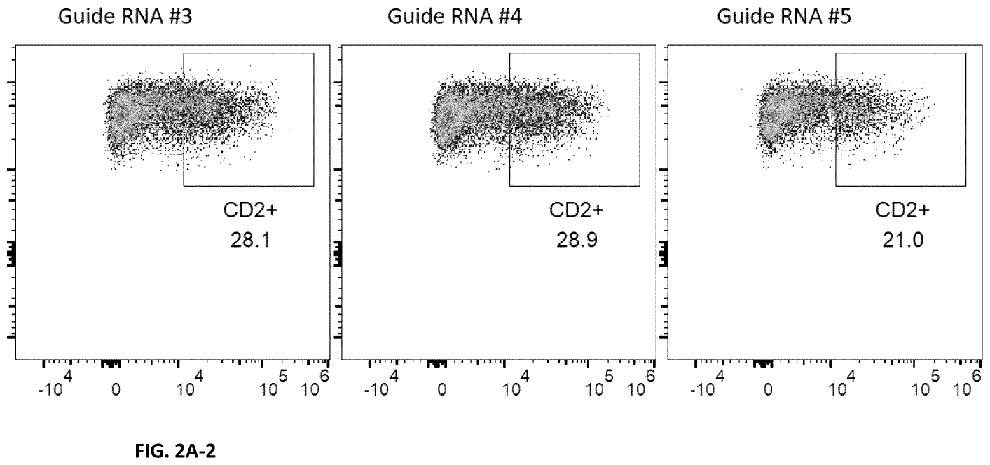

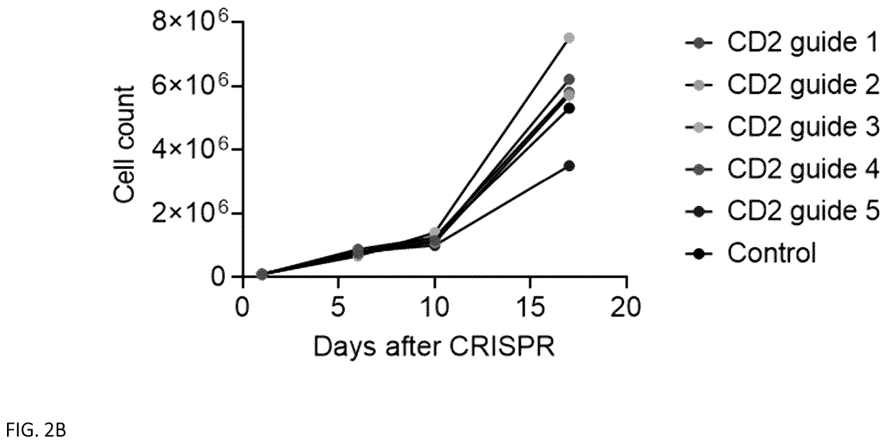

As shown in FIG. 2A, the removal of CD2 slowed down T cell proliferation when any of the five guide RNAs were used. Additionally, Tregs treated with all five guide RNAs were capable of expanding to high numbers (FIG. 2B).

### Example 3-CRISPR-Removal of CD2 Molecule Increases the Stability and Functionality of Tregs In Vitro

Using the methods in Example 1 and Guide #5 (SEQ ID NO: 5), it was found that the removal of CD2 from the Tregs increased their stability and functionality.

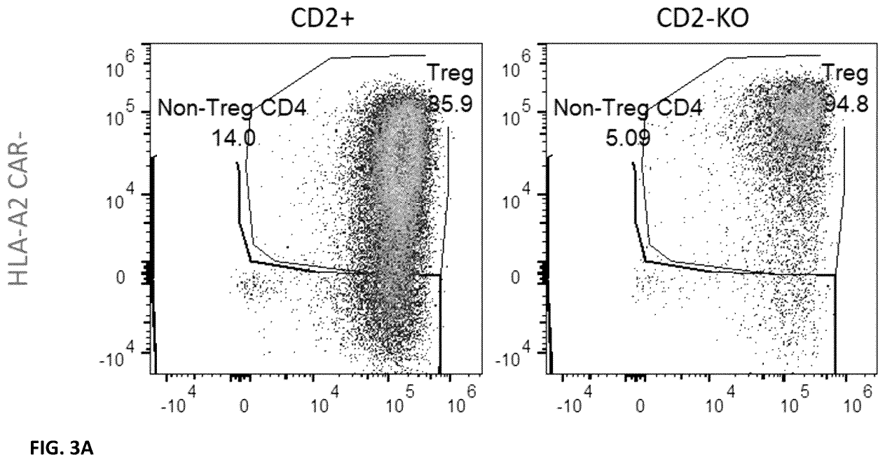

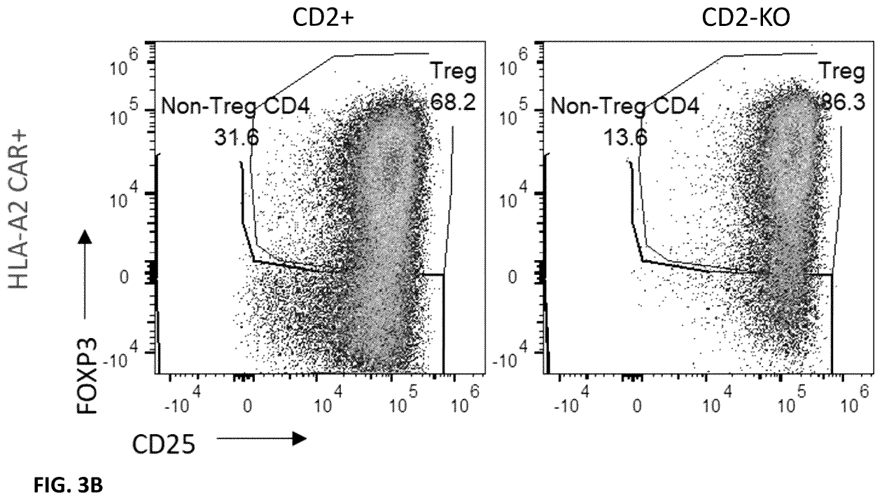

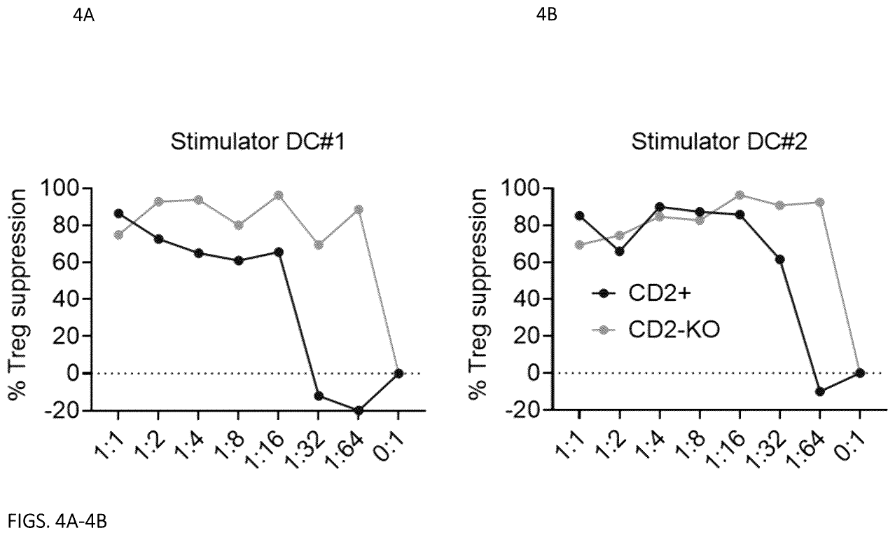

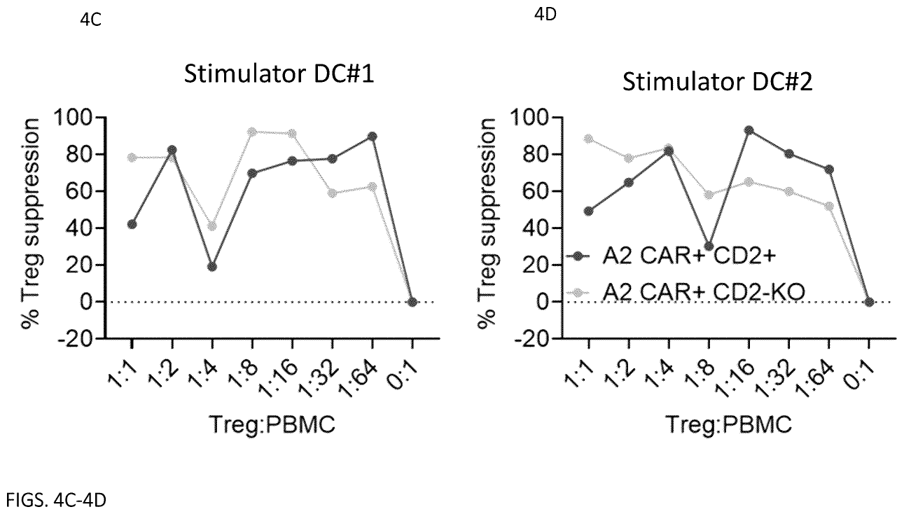

FIGS. 3 and 4 show the results of the Treg suppression assay. FIG. 3 shows representative flow cytometry of the Tregs with intact CD2 (CD+) and with the removal of CD2 (CD2−). As can be seen, the CD-cells are more stable as remaining Treg cells, whether they were HLA-A2-CAR+ or HLA-A2-CAR−. See FIG. 3.

FIG. 4 shows the results of flow cytometry and that the Treg cells with the CD2 removed were more stable, i.e., less likely to convert to non-Treg cells, whether they were HLA-A2-CAR+ (FIGS. 4C and 4D) or HLA-A2-CAR− (FIGS. 4A and 4B).

### Example 4—In Vivo Study Showed Higher Functionality of CD2-KO HLA-A2 CAR Tregs and CD2-KO Tregs in Preventing the Expansion of T Cells, Increasing Survival and Delaying the Development of GVHD

Using the protocol in Example 1 and shown in FIG. 5, four groups of Tg NSG mice were injected with 800K Tregs from the 4 Treg groups to prevent GVHD. A fifth control group was also used.

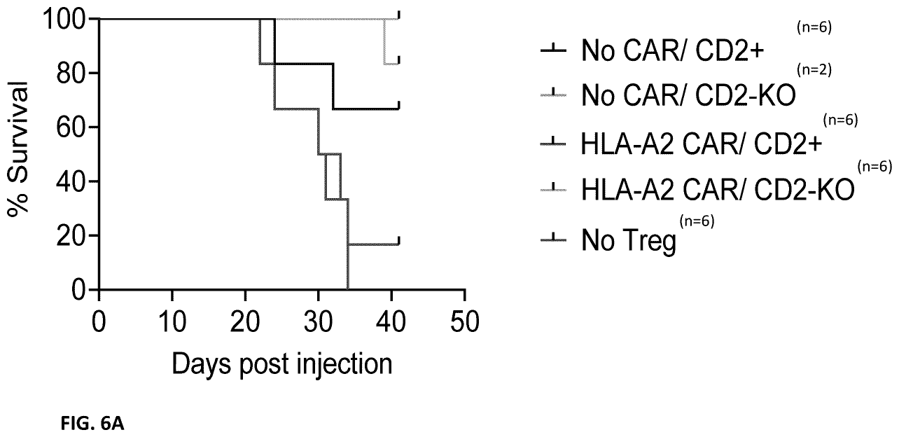

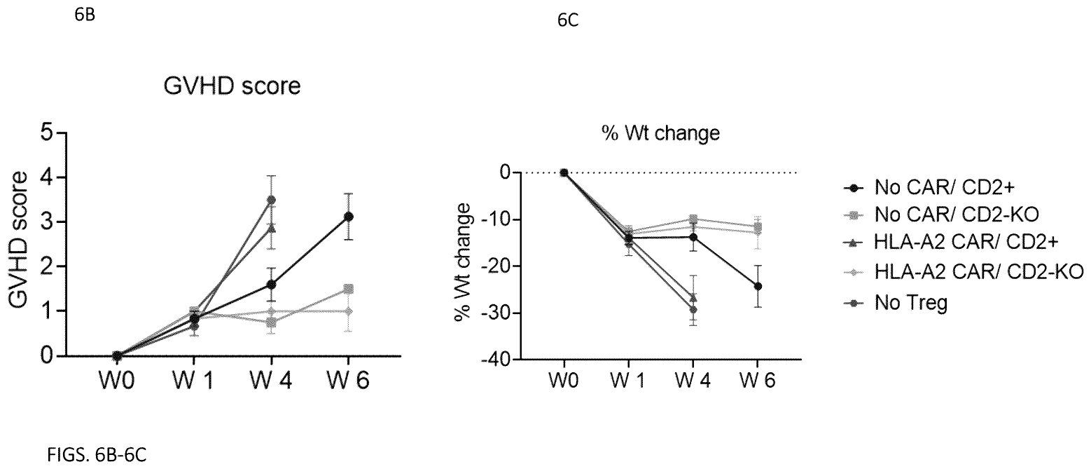

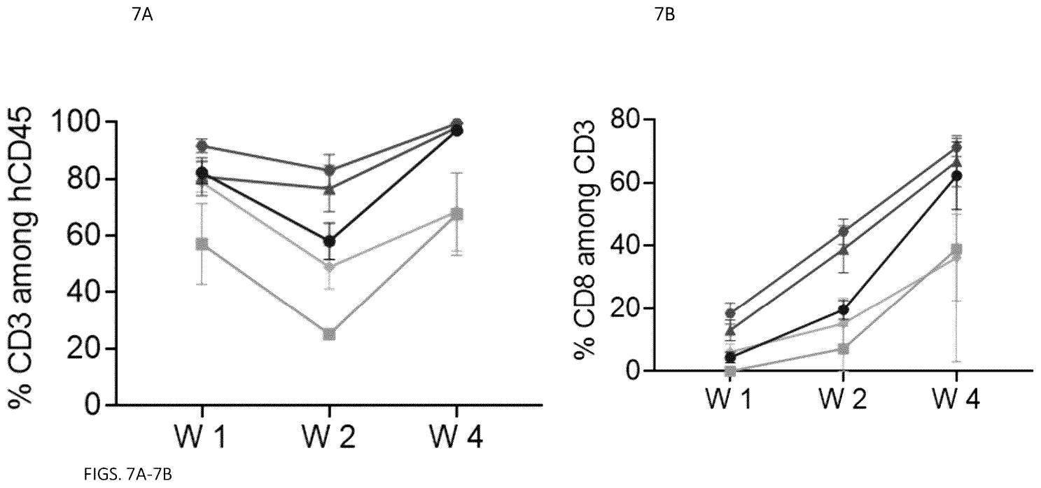

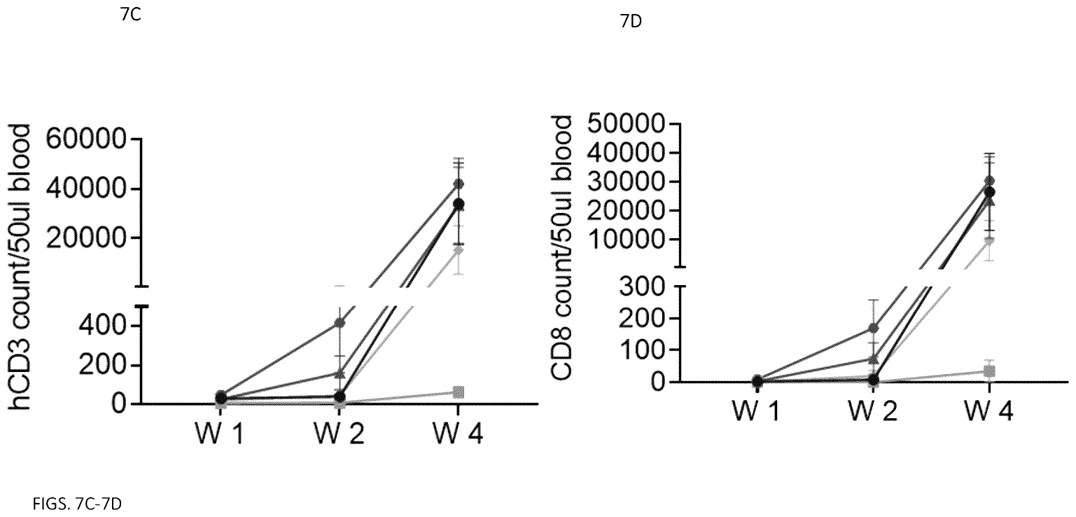

As shown in FIGS. 6 and 7, the in vivo testing showed higher functionality of the CD2− CAR Tregs at preventing the expansion of T cells and delaying the development of GVHD.

FIG. 6A is a survival curve which shows that the mice treated with CD2− cells had greater survival than mice treated with CD2+ cells, whether treated with CAR Tregs CD2− and Tregs CD2− with no CAR.

FIG. 6B is a graph of GVHD scores for the various groups of mice. The mice treated with the Treg CD2− cells, both CAR+ and CAR−, had the lowest GVHD score throughout the treatment, with the CAR Tregs CD2− cells staying at a consistently low score throughout the testing period.

FIG. 6C shows that the mice treated with the CD-cells, both CAR Tregs and Tregs alone, had very little weight change.

FIG. 7 shows the results of the analysis of immune cells in peripheral blood using flow cytometry.

### REFERENCES

Hua. et al. Pathological conversion of regulatory T cells is associated with loss of allotolerance. Sci. Rep. (2018). doi: 10.1038/s41598-018-25384-x
Koenen et al. Human CD25highFoxp3pos regulatory T cells differentiate into IL-17 producing cells. Blood (2008). doi: 10.1182/blood-2008-01-133967
Neelapu. CAR-T efficacy: Is conditioning the key? Blood (2019). doi: 10.1182/blood-2019-03-900928
Raffin et al. Treg cell-based therapies: challenges and perspectives. National review of Immunology (2020 March) 20 (3): 158-172. doi: 10.1038/s41577-019-0232-6. Epub 2019 Dec. 6.
Sadlon et al. Genome-wide Identification of Human FOXP3 Target Genes Cells in Natural Regulatory T Cells. Journal of Immunology (2010) 185 (2): 1071-1081.
Zhou et al. Instability of the transcription factor Foxp3 leads to the generation of pathogenic memory T cells in vivo. Nature Immunology (2009 September); 10 (9): 1000-7. Epub 2009 Jul. 26.

1. A regulatory T (Treg) cell engineered such that it is deficient in a cell-surface antigen, and resistant to conversion to an effector T cell (Teff) and resistant to the effect of T cell-depleting conditioning regimens.

2. The Treg cell of claim 1, further comprising a first nucleic acid construct encoding a chimeric antigen receptor (CAR), wherein the CAR comprises an antigen-binding region.

3. The Treg cell of claim 1, wherein the cell-surface antigen is CD2 and the T cell-depleting conditioning regimens involve a CD2 antibody.

4. The Treg cell of claim 1, wherein the entire or a portion of the gene encoding the cell-surface antigen is deleted from the Treg cell.

5. The Treg cell of claim 4, wherein the entire or a portion of the gene encoding the cell-surface antigen is deleted from the Treg cell using an RNA-guided nuclease and at least one guide RNA.

6. The Treg cell of claim 2, wherein the antigen-binding region is a single-chain variable fragment (scFv) comprising a light chain variable region (VL) and a heavy chain variable region (VH).

7. The Treg cell of claim 2, wherein the CAR binds to human leukocyte antigen A2 (HLA-A2) and is operably linked to a Treg-specific promoter.

8. A method of producing the Treg cell of claim 1, comprising introducing into a Treg cell: (i) at least one guide RNA (gRNA) or DNA encoding at least one guide RNA (gRNA), which hybridizes to a portion of the nucleotide sequence that encodes a cell-surface antigen; and (ii) at least one RNA-guided endonuclease or a nucleic acid encoding an RNA-guided endonuclease.

9. The method of claim 8, wherein the cell-surface antigen is CD2.

10. The method of claim 8, wherein the RNA-guided endonuclease is a Cas nuclease.

11. The method of claim 10, wherein the Cas nuclease is Cas9.

12. The method of claim 9, wherein the gRNA has a nucleotide sequence selected from the group consisting of SEQ ID NOs: 1-5.

13. The method of claim 8, wherein the at least one guide RNA and the RNA-guided endonuclease are introduced to the cell in a ribonucleoprotein complex.

14. A composition comprising the Treg cells of claim 1.

15. The composition of claim 14, further comprising a pharmaceutically acceptable carrier.

16. A method of inducing immune tolerance, or immunosuppression in a subject in need thereof, comprising administering to the subject the cell of claim 1.

17. A method of treating, reducing and/or preventing rejection or reducing complications of transplantation in a subject to a graft obtained from a donor mammal, comprising administering to the subject the cell of claim 1 before, during or after transplantation.

18. The method of claim 17, wherein the donor mammal is allogenic or xenogenic.

19. A method of treating or preventing an autoimmune disease or disorder in a subject in need thereof, comprising administering to the subject the cell of claim 1.

20. A method of treating or preventing graft-versus-host disease in a subject in need thereof, comprising administering to the subject the cells of claim 1.
